(19) United States
(12) Patent Application Publication
(10) Pub. No.: US 2009/0068585 A1
(43) Pub. Date: Mar. 12, 2009

(54) DISSOLUTION PROMOTER AND PHOTORESIST COMPOSITION INCLUDING THE SAME

(76) Inventors: Dong-Woo HAN, Hwaseong-Si (KR); Jeong-Sik Kim, Hwaseong,Si (KR); Min-Ja Yoo, Boryeong-Si (KR); Jae-Woo Lee, Bucheon-Si (KR); Jae-Hyun Kim, Seoul (KR)

Correspondence Address: BIRCH STEWART KOLASCH & BIRCH, PO BOX 747, FALLS CHURCH, VA, 22040-0747, US

(21) Appl. No.: 12/208,880

(22) Filed: Sep. 11, 2008

(30) Foreign Application Priority Data
Sep. 12, 2007 (KR) 10-2007-0092796

(51) Int. Cl. G03C 1/38; G03F 7/20; C07C 69/75; C07C 69/74; C07J 9/00; C07D 401/14

(52) U.S. Cl. 430/270.1; 430/325; 560/127; 560/122; 552/553; 540/145

(57) ABSTRACT

In the formation of a fine pattern using a photolithography process, a dissolution promoter which can increase the difference of solubility between exposed region and unexposed region, and a photoresist composition including the same are disclosed. The dissolution promoter has the structure of the following formula (wherein, R is a hydrocarbon group of 1 to 40 carbon atoms, A is an alkyl group of 1 to 10 carbon atoms, p is 0 or 1, and q is an integer of 1 to 20).

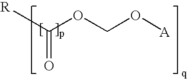

Moreover, the photoresist composition comprises 3 to 30 wt % (weight %) of the photosensitive compound; 1 to 30 weight parts of a dissolution promoter represented by the formula, with respect to 100 weight parts of the photosensitive compound; 0.05 to weight parts of a photo-acid generator with respect to 100 weight parts of the photosensitive compound; and a remaining organic solvent.

This application claims the priority benefit of Korean Patent Application No. 10-2007-0092796 filed on Sep. 12, 2007. All disclosure of the Korean Patent application is incorporated herein by reference.

### FIELD OF THE INVENTION

This invention relates to a dissolution promoter, and more particularly, in the formation of a fine pattern using a photolithography process, to a dissolution promoter which can increase the difference of solubility between exposed region and unexposed region, and a photoresist composition including the same.

### BACKGROUNDS OF THE INVENTION

Generally, a chemically amplified photoresist composition for a photolithography process, includes a photosensitive polymer whose solubility to developer is changed by reacting with acid, a photo acid generator which can generate acid by light exposing, and an organic solvent. The difference of solubility between exposed region and unexposed region is increased because the acid generated in the exposed region induces the deprotected reaction of a photosensitive polymer. The photoresist composition can further include a dissolution inhibitor insoluble to water or developer, to form a durable and gently sloped photoresist pattern by making unexposed region harder. However, the development of materials to dissolve exposed region more effectively is desired than the dissolving inhibitor which makes a durable polymer more durable.

### SUMMARY OF THE INVENTION

Therefore, it is an object of the present invention to provide a dissolution promoter which can effectively increase the difference of solubility between exposed region and unexposed region, and a photoresist composition including the same.

It is another object of the present invention to provide a dissolution promoter which can increase the solubility of polymer to developer, and be deprotected with a little amount of acid, and a photoresist composition including the same.

It is still another object of the present invention to provide a method for forming the photoresist pattern which can increase the contrast of photoresist pattern, and prevent the formation of various defects and bridges during development process, and improve the line edge roughness.

In order to achieve these objects, the present invention provides a dissolution promoter represented by the following Formula 1.

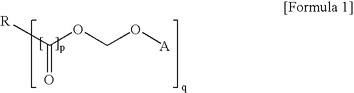

In the Formula 1, R is a hydrocarbon group of 1 to 40 carbon atoms, A is an alkyl group of 1 to 10 carbon atoms, p is 0 or 1, and q is an integer of 1 to 20.

The present invention also provides a photoresist composition comprising 3 to 30 wt % (weight %) of the photosensitive compound; 1 to 30 weight parts of a dissolution promoter represented by the Formula 1 with respect to 100 weight parts of the photosensitive compound; 0.05 to 10 weight parts of a photo-acid generator with respect to 100 weight parts of the photosensitive compound; and a remaining organic solvent. Moreover, the present invention provides a method for forming photoresist pattern composition comprising the step of: (a) coating the above-mentioned photoresist composition on a substrate to form a photoresist layer; (b) exposing the photoresist layer to a light; (c) heating the exposed photoresist layer; and (d) developing the heated photoresist layer to form the photoresist pattern.

### BRIEF DESCRIPTION OF THE DRAWINGS

FIG. 1 shows the result of the 1H-NMR test to check the deprotecting reaction of the dissolving promoter in the present invention.

FIG. 2 shows the result of the IR test to check the deprotecting reaction of the dissolving promoter in the present invention.

FIG. 3 to 6 show an electron microphotograph of the photoresist pattern formed by using a photoresist composition according to the examples and comparative example of the present invention.

### DETAILED DESCRIPTION OF THE INVENTION

A more complete appreciation of the invention, and many of the attendant advantages thereof, will be better appreciated by reference to the following detailed description.

The dissolution promoter according to the present invention, is used as one component of a photoresist composition, and is represented by the following Formula 1.

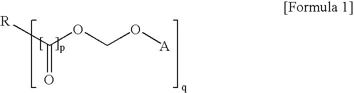

In the Formula 1, R is a hydrocarbon group of 1 to 40, preferably 4 to 30, more preferably 5 to 20 carbon atoms, preferably, a hydrocarbon group comprising cycloalkyl group or multi cycloalkyl group, more preferably, cycloalkyl group or multi cycloalkyl group. If necessary, R can be a ring structure comprising a hetero atom such as a nitrogen atom (N), a phosphorus atom (P), a sulfur atom (s), and an oxygen atom (O). A is an alkyl group of 1 to 10, preferably 1 to 4 carbon atoms, and if necessary, is capable of comprising a hetero atom such as an oxygen atom (O), for example, —CH₃, —CH₂CH₃, —CH₂CH₂OCH₃, and —CH₂CH₂OCH₂CH₃. p is 0 or 1, and q is an integer of 1 to 20 preferably 2 to 10, more preferably 2 to 5. Moreover, the R and A may be substituted with a substituent such as an alkyl group of 1 to 4 carbon atoms, an alkoxy group of 1 to 4 carbon atoms, and halogen atom, or may not be substituted.

The representative examples of the dissolution promoter represented by the Formula 1 include

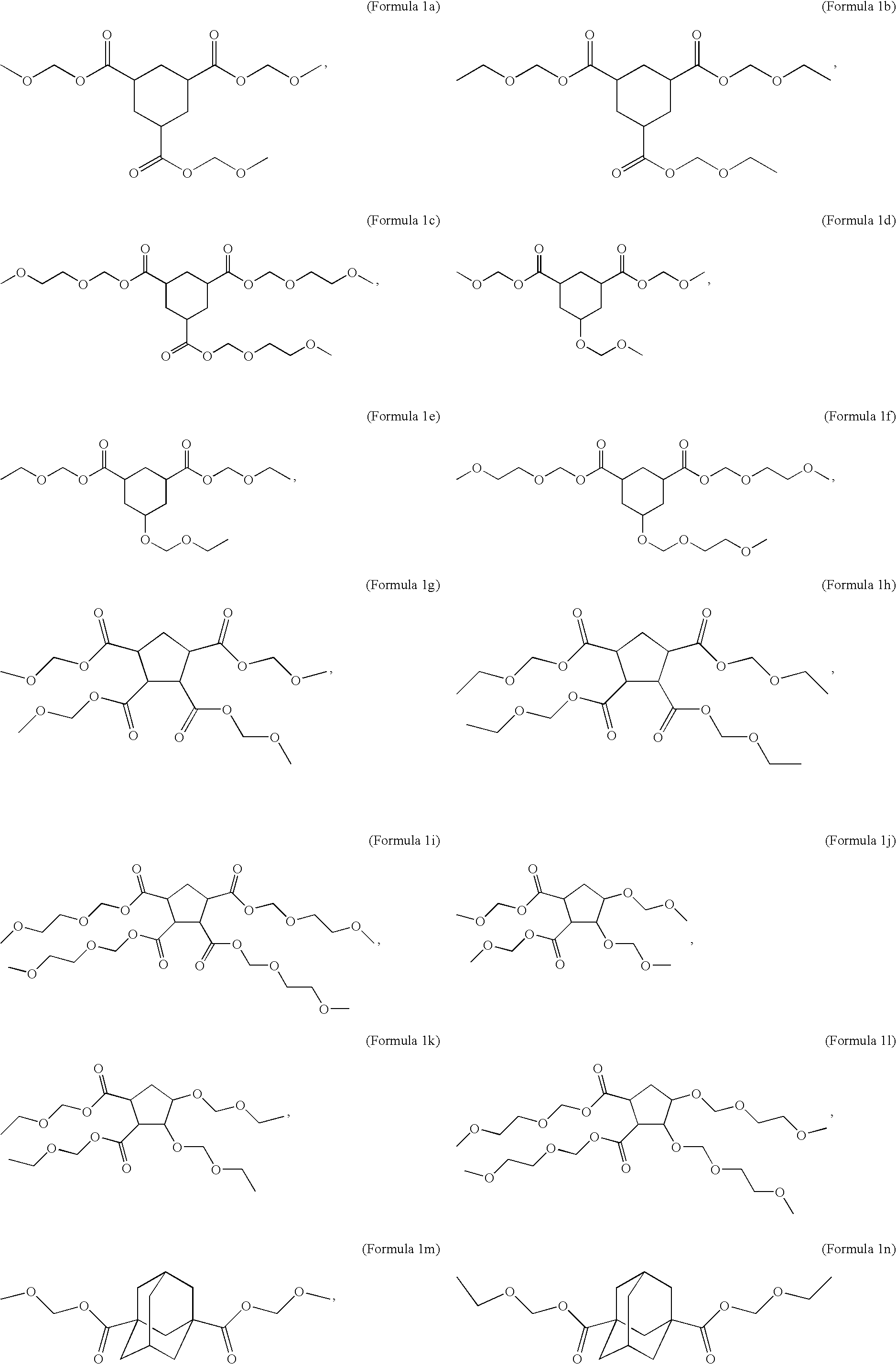

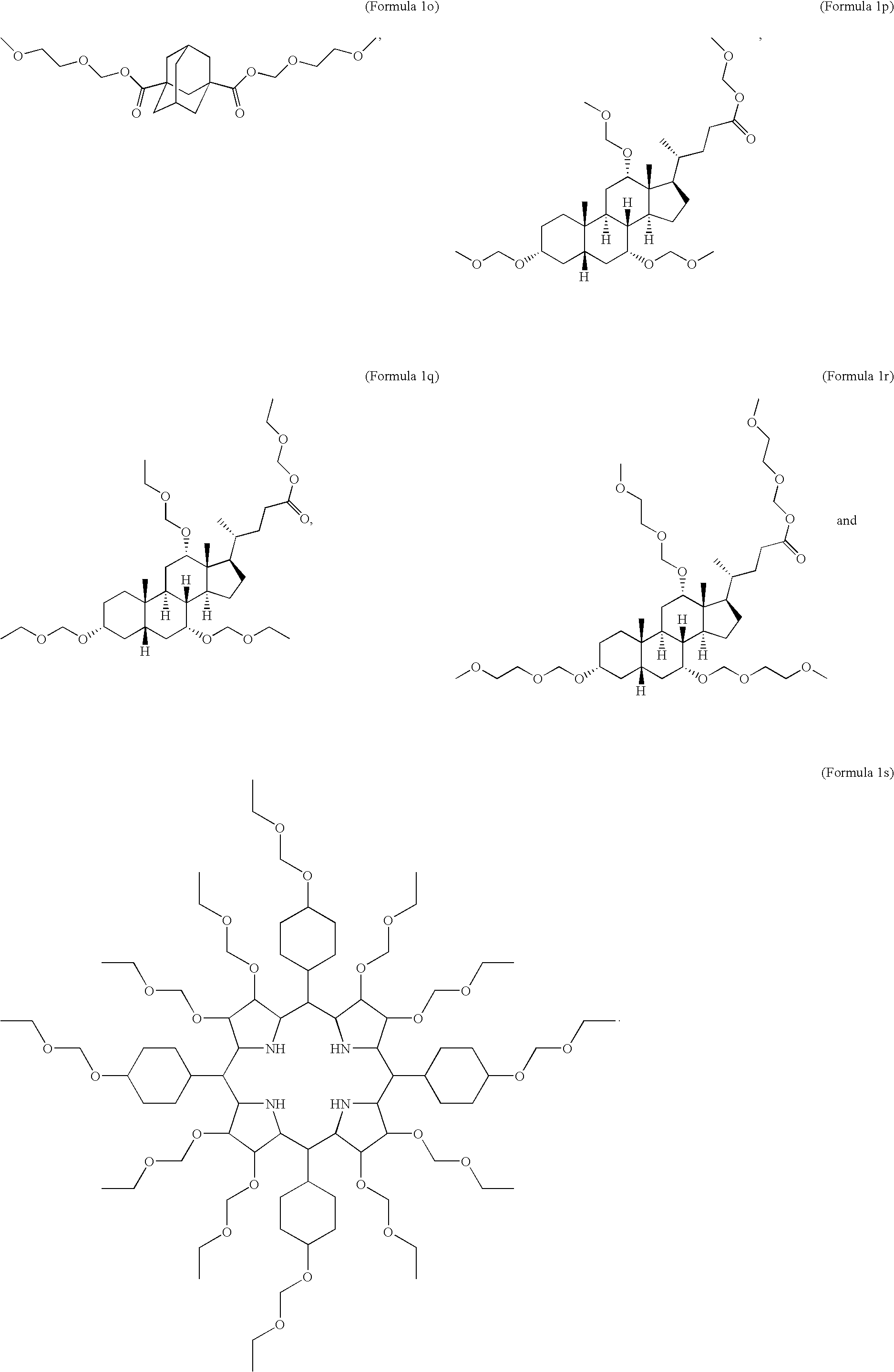

The dissolution promoter is a compound which protects alcohol or organic acid (carboxylic acid) groups by acetalization. For example, as described in the following Reaction 1, the dissolution promoter is synthesized by reacting a compound including alcohol or carboxylic acid with an alkyl halide at room temperature, and is purified by recrystallization with hexane or column chromatography with hexane and ethyl acetate after several times of washing a base by water. The following reaction enables much of the dissolution promoter to be easily and fast obtained because it is easier than the conventional acetal synthesizing reaction using ketone.

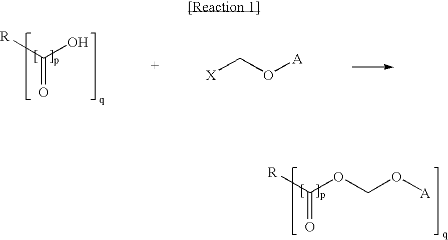

In the Reaction 1, R, A, p and q are the same as defined in the Formula 1, and X is a halogen atom such as Cl, Br and I.

The alkyl halide used in the Reaction 1 includes

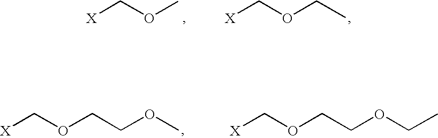

and so on (wherein, X is a halogen atom such as Cl, Br and I). A reactant which is able to produce a dissolution promoter by reacting with the alkyl halide, includes compounds represented by the following Formula 2.

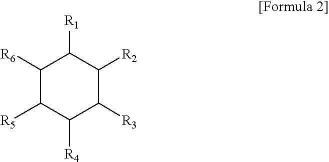

In the Formula 2, Y is a carbon atom, a nitrogen atom, a phosphorus atom, sulfur atom or an oxygen atom, and the cyclic structure of the body can be a hetero cyclic structure such as piperidine. R₁, R₂, R₃, R₄, R₅ and R₆ are independently, a hydrogen atom, a hydroxyl group or a carboxyl group, and at least one of R₁ to R₆ is a hydroxyl group or a carboxyl group. Wherein, if Y is a sulfur atom or an oxygen atom, R₂ does not exist.

The representative examples of the reactant represented by the Formula 2 include

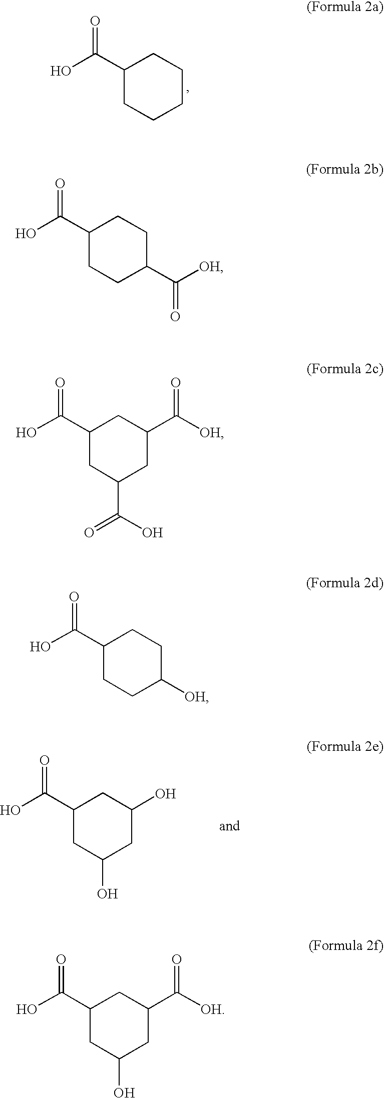

Another example of a reactant which is able to produce a dissolution promoter, includes the compound represented by the Formula 3.

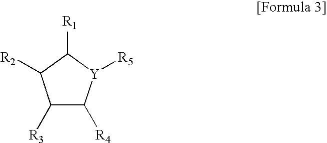

In the Formula 3, Y is a carbon atom, a nitrogen atom, a phosphorus atom, a sulfur atom or an oxygen atom, and the cyclic structure of the body can be a hetero cyclic structure such as pyrrolidine. R₁, R₂, R₃, R₄ and R₅ are independently, a hydrogen atom, a hydroxyl group or a carboxyl group, and at least one of R₁ to R₅ is a hydroxyl group or a carboxyl group. Wherein, if Y is a sulfur atom or an oxygen atom, R₅ does not exist.

The representative examples of the reactant represented by the Formula 3 include

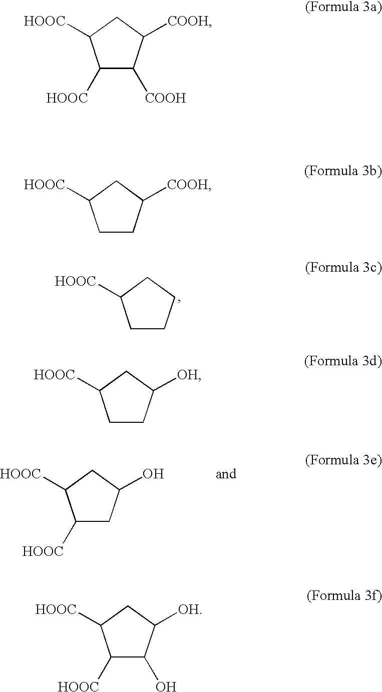

Still another example of a reactant which is able to produce a dissolution promoter, includes the compound represented by the Formula 4.

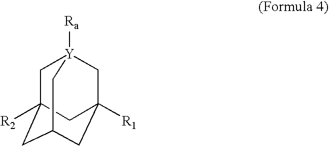

In the Formula 4, Y is a carbon atom, a nitrogen atom or a phosphorus atom, and the cyclic structure of the body can be a hetero adamantane structure R₁, R₂ and R₃ are independently, a hydrogen atom, a hydroxyl group or a carboxyl group, and at least one of R₁ to R₃ is a hydroxyl group or a carboxyl group. Wherein, if Y is a nitrogen atom or a phosphorus atom, R₃ does not exist.

The representative examples of the reactant represented by the Formula 4 include

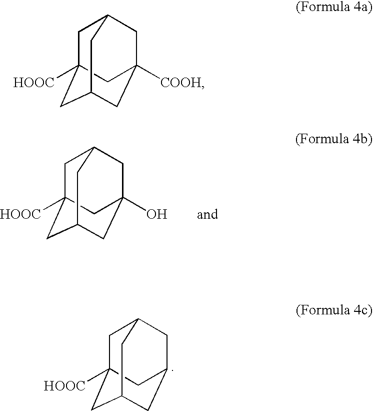

Still another example of a reactant which is able to produce a dissolution promoter, includes the compound represented by the Formulas 5a to 5e.

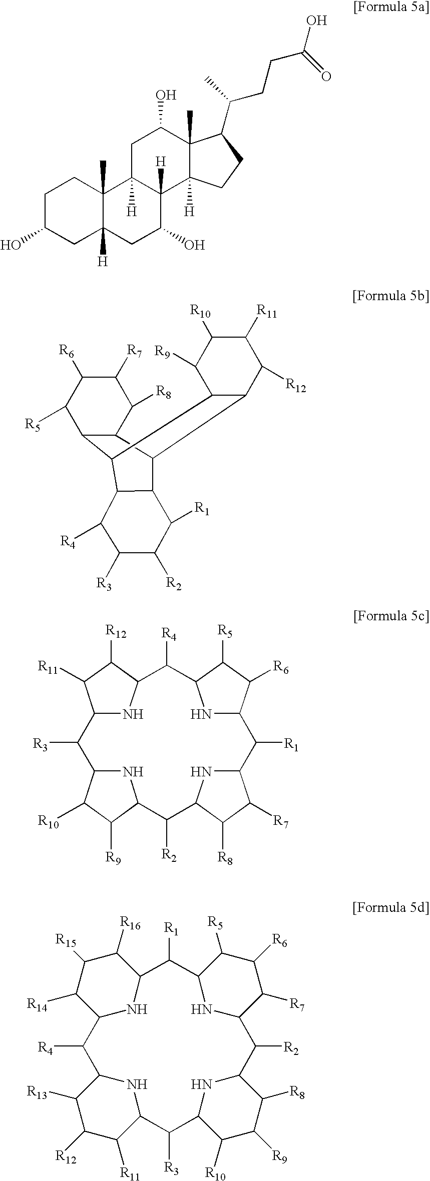

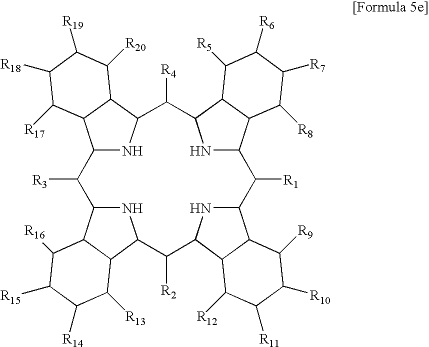

In the Formulas 5a to 5e, R₁ to R₂₀ are independently, a hydrogen atom, a hydroxyl group or a carboxyl group, or an alkyl group of 1 to 10 carbon atoms preferably a cycloalkyl group of 5 to 10 carbon atoms, comprising a hydroxyl group or a carboxyl group. At least one of R₁ to R₂₀ is a hydroxyl group, a carboxyl group or an alkyl group of 1 to 10 carbon atoms, preferably a cycloalkyl group of 5 to 10 carbon atoms, comprising a hydroxyl group or a carboxyl group. The representative examples of the reactant represented by the Formulas 5b to 5e include

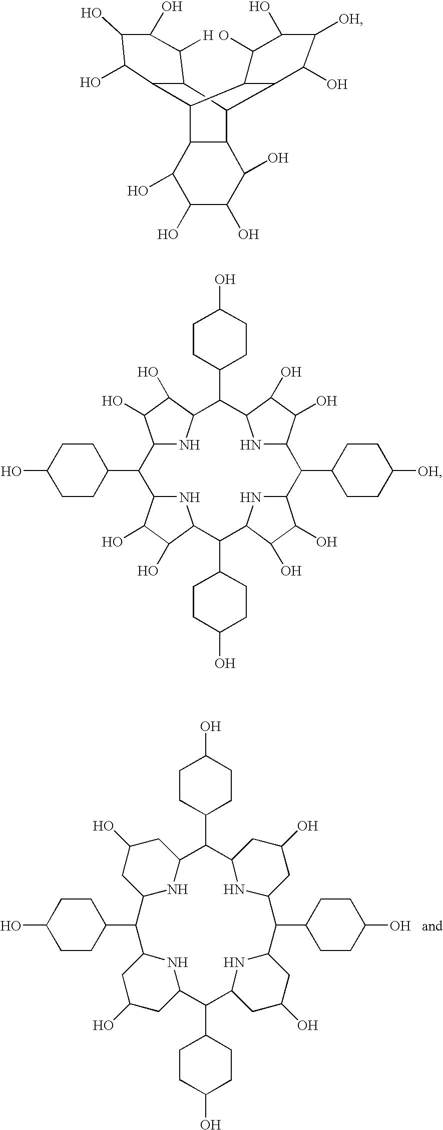

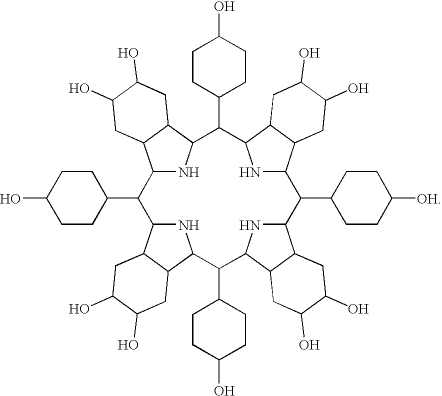

Like this, as the reactant which is able to produce a dissolution promoter of the present invention, various organic acids, multiple synthesis connected ring structure or a macro cyclic molecule, which comprise at least one of a hydroxyl group or a carboxyl group can be used.

The dissolution promoter of the present invention remarkably improves solubility of the exposed region even with a little acid, because it has simple structure and small volume as well as many acetal-like protecting groups as the activation sites easily deprotected by acid. Concretely, with the dissolution promoter of the present invention, acid used for forming patterns under light exposure improves solubility of the exposed region by leading a deacetylization of the dissolution promoter during development. On the other hand, there is no acid generation and solubility wouldn't be improved on unexposed region, and the solubility difference between exposed region and unexposed region becomes broad. Moreover, in the case of using the dissolution promoter of the present invention, carboxylic acids or alcohols generated from the dissolution promoter can solve and easily remove polymer-pieces which are able to act as defect on the interface between exposed region and unexposed region. Moreover, the dissolution promoter of the present invention is distinguished for being more easily solved and developed by general developers such as 2.34% Tetramethyl ammonium hydroxide (TMAM). The deprotecting reaction mechanism of the dissolution promoter in an aqueous solution can be explained by the following Formula 2 which is the general SN1 type formula. That is, after the light exposure under desired light, the reaction is happened in developer or pure water during the developing process. Acid generated during light exposure remains as an alcohol cation intermediate by accepting an electron from an oxygen atom of acetal, and is slowly detached as an alcohol molecule leaving a carbon cation which then accepts an electron from a water molecule. Then the water molecule becomes an alcohol by donating a hydrogen atom which is then attacked by another oxygen atom of the acetal and detached as another alcohol. The regenerated acid is removed with water just after development. Therefore, the deprotecting reaction of acetal is possible even with catalytic quantity of acid in an aqueous solution, and solvency can be improved with still less quantity of acid than in previous deprotecting reactions. Moreover, the efficiency of the dissolution promoter can be increased and decreased freely according to various conditions by designing many reactive sites in the dissolution promoter molecule because one reacting site generates two types of alcohol by deprotecting reaction.

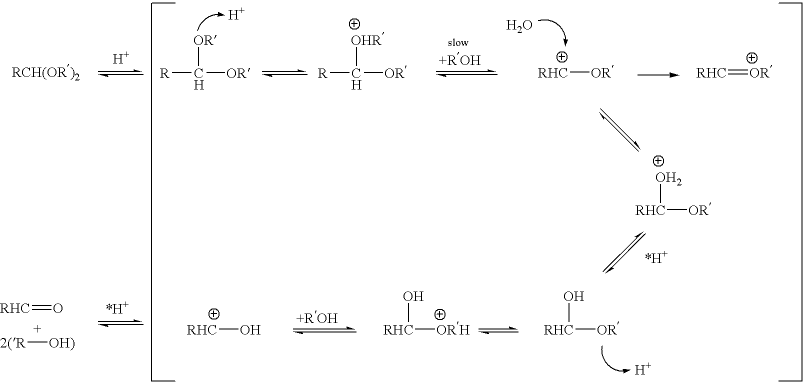

The photoresist composition according to the present invention includes the dissolution promoter represented by the Formula 1, a photosensitive polymer, a photo-acid generator and an organic solvent, and, if necessary, further includes a base compound as a quencher, and a surfactant. In the photoresist composition, the amount of the photosensitive polymer is 3 to 30 wt %, preferably 3 to 15 wt %. Moreover, the dissolution promoter represented by the Formula 1 is 1 to 30 weight parts, preferably 2 to 10 weight parts with respect to 100 weight parts of the photosensitive polymer, the amount of the photo-acid generator is 0.05 to 10 weight parts with respect to 100 weight parts of the photosensitive polymer, and the rest component of the photoresist composition is the organic solvent. Moreover, the amount of the base compound, if used, is 0.01 to 10 wt %, preferably 0.01 to 2 wt % with respect to the total photoresist composition. Wherein, if the amount of the dissolution promoter is too little (less than 1 weight parts), it is difficult to increase the difference of solubility between exposed region and unexposed region effectively, and the purpose of the present invention is beyond attainment. If the amount of the dissolution promoter is too much (more than 30 weight parts), properties of polymer compounds, main components of photoresist, are changed and acid generated during light exposure can inhibit the deprotecting reaction of the polymers, which ultimately damages the resolving power of photoresist. Moreover, increase in the content of small molecules makes the solubility of polymer compounds increased, and even unexposed region can be dissolved during developing process after light exposure. Moreover, if the amount of the photosensitive polymer is too little (less than 3 wt %), it is difficult to form the resist film with a desired thickness. If the amount of the photosensitive polymer is too much (more than 30 wt %), thickness of patterns on the wafer is not uniform. If the amount of the base compound is too little (less than 0.01 weight parts), it is not easy to control a diffusion of the acid generated in an exposure process so that the pattern profile is uneven. If the amount of the base compound is too much (more than 10 weight parts), the diffusion of the acid generated is suppressed so that pattern is not easily formed. And, if the amount of the PAG is too little (less than 0.05 weight parts), the light sensitivity of the photoresist composition may decrease. if the amount of the PAG is too much (more than 10 weight parts), the profile of the photoresist patterns may be deteriorated because the PAG absorbs a lot of ultraviolet rays and a large quantity of acid is produced from the PAG.

As the photosensitive polymer, any conventional photosensitive polymer for the photoresist, which reacts with an acid to vary solubility to a developer, can be used. The photosensitive polymer, which has a protecting group which is sensitive to an acid and then is separated by the acid, can be preferably used. The photosensitive polymer may be block copolymer or random copolymer, and the weight average molecular weight (Mw) of photosensitive polymer is preferably 3,000 to 20,000. As the PAG, any conventional PAG, which can generate an acid when exposed to light, can be used. The non-limiting examples of the PAG include onium salts, for example sulfonium salts or iodonium salts. Specifically, the PAG is selected from a group consisting of phthalimidotrifluoromethane sulfonate, dinitrobenzyltosylate, n-decyl disulfone and naphthylimido trifluoromethane sulfonate. Moreover, the PAG is selected from the group consisting of diphenyl iodonium triflate, diphenyl iodonium nonaflate, diphenyl iodonium hexafluorophosphate, diphenyl iodonium hexafluoroarsenate, diphenyl iodonium hexafluoroantimonate, diphenyl p-methoxyphenyl sulfonium triflate, diphenyl p-toluenyl sulfonium triflate, diphenyl p-tert-butylphenyl sulfonium triflate, diphenyl p-isobutylphenyl sulfonium triflate, triphenylsulfonium triflate, tris(p-tert-butylphenyl) sulfonium triflate, diphenyl p-methoxyphenyl sulfonium nonaflate, diphenyl p-toluenyl sulfonium nonaflate, diphenyl p-tert-butylphenyl sulfonium nonaflate, diphenyl p-isobutylphenyl sulfonium nonaflate, triphenylsulfonium nonaflate, tris(p-tert-butylphenyl) sulfonium nonaflate, triphenylsulfonium hexafluoroarsenate, triphenylsulfonium hexafluoroantimonate, triphenylsulfonium triflate and dibutylnaphtylsulfonium triflate.

The conventional various organic solvents for the photoresist composition can be used as the organic solvent of the photoresist composition of the present invention. Exemplary organic solvent include, but are not limited to, ethyleneglycol monomethylethyl, ethyleneglycol monoethylether, ethyleneglycol monomethylether, ethyleneglycol monoacetate, diethylene glycol, diethyleneglycol monoethylether, propyleneglycol monomethyletheracetate (PGMEA), propyleneglycol, propyleneglycol monoacetate, toluene, xylene, methylethylketone, methyl isoamyl ketone, cyclohexanone, dioxane, methyl lactate, ethyl lactate, methyl pyruvate, ethyl pyruvate, methyl methoxy propionate, ethyl ethoxy propionate, N,N-dimethylformamide, N,N-dimethylacetamide, N-methyl-2-pyrollidone, 3-ethoxy ethyl propionate, 2-heptanone, [symbol] -butyrolactone, ethyl 2-hydroxy propionate, ethyl 2-hydroxy-2-methyl propionate, ethoxyethyl acetate, hydroxylethyl acetate, methyl 2-hydroxy-3-methylbutanoate, methyl 3-methoxy-2-methylpropionate, ethyl 3-ethoxy propionate, ethyl 3-methoxy-2-methyl propionate, ethyl acetate, butyl acetate and mixture thereof. Moreover, the base compound as the quencher includes tri-ethylamine, tri-iso-butylamine, tri-iso-octylamine, di-ethanolamine, tri-ethanolamine and mixture thereof. It is obvious that conventional PAG, solvent and base may be used except the above mentioned PAG, solvent and base.

The surfactant, at need, is added in the present photoresist composition so as to improve a mixing uniformity of the photoresist composition, coating property of the photoresist composition and developing property of the photoresist film after the exposure to light. As the surfactant, any conventional surfactant, which can be used in the photoresist composition, can be used. Examples of such surfactants include fluorine-based surfactant or fluorine-silicon-based surfactant. The amount of the surfactant is 0.001 to 2 weight parts, preferably 0.01 to 1 weight parts with respect to solid content 100 weight parts of the photoresist composition. If the amount of the surfactant is too little, function of surfactant does not sufficiently work, and if the amount of the surfactant is too much, the resist property such as shape stability or a storage stability of the composition except for the coating property, may be adversely affected.

In order to form a photoresist pattern with the photoresist composition according to the present invention, first, the photoresist is applied or coated on a substrate such as silicon wafer, an aluminum substrate, and so on, for example, with a spin coater to form a photoresist layer. The photoresist layer is exposed to a light of a predetermined pattern. After the exposure, if necessary, the photoresist pattern is thermally treated (heated), which is called as PEB (Post Exposure Bake), and is developed to form the photoresist pattern. As the developing solution for the developing process, an alkali aqueous solution including an alkali compound such as sodium hydroxide, potassium hydroxide, sodium carbonate, tetramethylammonium hydroxide (TMAH) of the concentration of 0.1 to 10 weight % can be used. If necessary, the developing solution may further include water-soluble organic solvent such as methanol, ethanol, and a surfactant of a proper amount.

Hereinafter, examples and comparative examples are provided to illustrate the present invention in more detail, but the present invention is not restricted or limited by the following examples and comparative examples.

### EXAMPLES 1 TO 19

### Preparation of Dissolution Promoter

The reactant 1 having the component and amount (0.1 mole) listed in the following Table 1 was added into a 250 ml round reaction flask, and dissolved by 100 ml of tetrahydrofuran (hereinafter, THF) as the reaction solvent. The temperature of the reaction solution was maintained at 0° C., and not to occur vigorous dehydration, sodium hydride (60% in Oil) was slowly added with caution. After 5 minutes, the reaction solution was stirred during 30 minutes at the room temperature. And the solution composed by Reactant 2 having the component and amount listed in the following Table 1 and 10 ml of THF was slowly dropped. In the Reactant 2 of the Table 1, compound A is

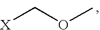

compound B is

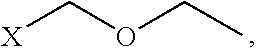

compound C is

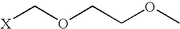

(wherein, X is a halogen atom (Cl)). After dropping the Reactant 2, the reaction solution was stirred for 12 hours at 40° C. After the reaction is finished, saturated sodium bicarbonate aqueous solution was slowly added for the neutral pH. Thereafter, 100 ml of cold distilled water was added in the reaction solution, and extraction was carried out 3 times with 200 ml of ethyl acetate. After the extracted solution was dried with anhydrous magnesium sulfate and distilled under the reduced pressure, the recrystallization with hexane or column chromatography with hexane and ethyl acetate by the ratio where Rf is 0.2 on TLC (Thin Layer Chromatography), was carried out to obtain the dissolution promoter having the component and yield listed in the following Table 1.

TABLE 1

| Reactant 1 and the amount used | Reactant 2 and the amount used | Product and yield |
|---|---|---|
| Compound 2c, 21.62 (g) | Compound A, 26.57 (g) (0.33 mol) | Formula 1a, 26.13 g (75%) |
| Compound 2c, 21.62 (g) | Compound B, 31.20 (g) (0.33 mol) | Formula 1b, 31.63 g (81%) |
| Compound 2c, 21.62 (g) | Compound C, 41.11 (g) (0.33 mol) | Formula 1c, 39.40 g (82%) |
| Compound 2f, 18.82 (g) | Compound A, 26.57 (g) (0.33 mol) | Formula 1d, 26.91 g (84%) |
| Compound 2f, 18.82 (g) | Compound B, 31.20 (g) (0.33 mol) | Formula 1e, 32.62 g (90%) |
| Compound 2f, 18.82 (g) | Compound C, 41.11 (g) (0.33 mol) | Formula 1f, 42.08 g (93%) |
| Compound 3a, 24.62 (g) | Compound A, 35.42 (g) (0.44 mol) | Formula 1g, 27.45 g (65%) |
| Compound 3a, 24.62 (g) | Compound B, 41.60 (g) (0.44 mol) | Formula 1h, 34.45 g (72%) |
| Compound 3a, 24.62 (g) | Compound C, 54.81 (g) (0.44 mol) | Formula 1i, 44.30 g (74%) |
| Compound 3f, 19.02 (g) | Compound A, 35.42 (g) (0.44 mol) | Formula 1j, 28.58 g (78%) |
| Compound 3f, 19.02 (g) | Compound B, 41.60 (g) (0.44 mol) | Formula 1k, 35.49 g (84%) |
| Compound 3f, 19.02 (g) | Compound C, 54.81 (g) (0.44 mol) | Formula 1l, 49.38 g (91%) |
| Compound 4a, 22.43 (g) | Compound A, 17.71 (g) (0.22 mol) | Formula 1m, 27.18 g (87%) |
| Compound 4a, 22.43 (g) | Compound B, 20.80 (g) (0.22 mol) | Formula 1n, 31.32 g (92%) |
| Compound 4a, 22.43 (g) | Compound C, 27.41 (g) (0.22 mol) | Formula 1o, 38.05 g (95%) |
| Compound 5a, 40.86 (g) | Compound A, 35.42 (g) (0.44 mol) | Formula 1p, 37.43 g (64%) |
| Compound 5a, 40.86 (g) | Compound B, 41.60 (g) (0.44 mol) | Formula 1q, 44.86 g (70%) |
| Compound 5a, 40.86 (g) | Compound C, 27.41 (g) (0.44 mol) | Formula 1r, 38.83 g (65%) |
| Compound 5c, 85.32 (g) | Compound B, 124.8 (g) (1.32 mol) | Formula 1s, 91.60 g (59%) |

The results of 1H-NMR and Td (Decomposed Temperature: The temperature at the break of the weakest bond in a molecule. Wherein, it is the temperature at the break of C—O bond in acetal, and was checked by the graph indicating change of molecular weight as the break of the weakest bond. Using Td, the stability during the exposing process can be checked, and generally, it is desired that Td is more than 250° C.) of the dissolution promoters obtained by the above reaction, are as follows.

The compound of Formula 1a: 1H NMR(CDCl₃, 400 MHz) 6.16 (s, 6H), 3.24 (s, 9H), 2.27 (t, J=7.0 Hz, 3H), 2.14 (d, J=6.9 Hz, 6H). Td: 297° C.

The compound of Formula 1b: 1H NMR(CDCl₃, 400 MHz) 6.20 (s, 6H), 3.41 (m, 6H), 2.35 (t, J=6.9 Hz, 3H), 2.14 (d, J=7.0 Hz, 6H), 1.11 (t, J=7.0 Hz, 9H). Td: 308° C.

The compound of Formula 1c: 1H NMR(CDCl₃, 400 MHz) 6.25 (s, 6H), 3.60˜3.70 (m, 6H), 3.50˜3.40 (m, 6H), 3.24 (s, 9H), 2.10 (t, J=6.9 Hz, 3H), 2.05 (d, J=6.9 Hz, 6H). Td: 317° C.

The compound of Formula 1d: 1H NMR(CDCl₃, 400 MHz) 6.20 (s, 4H), 5.45 (s, 2H), 3.33 (s, 6H), 3.23 (s, 3H), 2.95˜2.85 (m, 1H), 2.40˜2.35 (m, 1H), 2.10 (t, J=5.3 Hz, 2H), 1.95˜1.85 (m, 4H). Td: 286° C.

The compound of Formula 1e: 1H NMR(CDCl₃, 400 MHz) 6.20 (s, 4H), 5.40 (s, 2H), 3.55˜3.45 (m, 4H), 3.25˜3.20 (m, 2H), 2.90˜2.85 (m, 1H), 2.35˜2.25 (m, 2H), 2.07(t, J=5.0 Hz, 2H), 1.85 (t, J=5.0 Hz, 4H), 1.25˜1.15 (m, 6H), 1.10˜1.00 (m, 3H), Td: 303° C.

The compound of Formula 1f: 1H NMR(CDCl₃, 400 MHz) 6.25 (s, 4H), 5.40 (s, 2H), 3.50˜3.30 (m, 2H), 3.25 (bs, 6H), 3.20 (s, 3H), 2.90˜2.85 (m, 1H), 2.35˜2.25 (m, 2H), 2.22 (t, J=5.0 Hz, 2H), 1.90 (t, J=5.0 Hz, 4H). Td: 321° C.

The compound of Formula 1g: 1H NMR(CDCl₃, 400 MHz) 6.30˜6.20 (m, 8H), 3.55 (d, J=7.0 Hz, 2H), 3.30˜3.20 (m, 12H), 3.20˜3.00 (m, 2H), 2.22 (t, J=7.0 Hz, 2H). Td: 279° C.

The compound of Formula 1h: 1H NMR(CDCl₃, 400 MHz) 6.30˜6.20 (m, 8H), 3.57 (d, J=7.0 Hz, 2H), 3.40˜3.30 (m, 8H), 3.00˜2.90 (m, 2H), 2.24 (t, J=7.0 Hz, 2H), 1.10˜0.90 (m, 12H). Td. 290° C.

The compound of Formula 1i: 1H NMR(CDCl₃, 400 MHz) 6.30˜6.20 (m, 8H), 3.65 (d, J=6.8 Hz, 2H), 3.55˜3.45 (m, 16H), 3.20˜3.10 (m, 12H), 3.00˜2.90 (m, 2H), 2.21 (t, J=7.0 Hz, 2H). Td: 304° C.

The compound of Formula 1j: 1H NMR(CDCl₃, 400 MHz) 6.25 (s, 4H), 5.49 (s, 4H), 3.92 (t, J=4.9 Hz, 1H), 3.37 (t, J=5.0 Hz, 1H), 3.30˜3.20 (m, 12H), 3.00 (m, 1H), 2.90˜2.85 (m, 1H). Td: 287° C.

The compound of Formula 1k: 1H NMR(CDCl₃, 400 MHz) 6.19 (s, 4H), 5.45 (s, 4H), 3.85 (t, J=5.0 Hz, 1H), 3.30 (t, J=4.9 Hz, 1H), 3.45˜3.35 (m, 8H), 3.25˜3.20 (m, 1H), 3.00˜2.95 (m, 1H), 1.95 (t, J=7.0 Hz, 2H), 1.15˜0.95 (m, 12H). Td: 288° C.

The compound of Formula 1l: 1H NMR(CDCl₃, 400 MHz) 6.27 (s, 4H), 5.41 (s, 4H), 3.50˜3.30 (m, 16H), 4.00˜3.90 (m, 1H), 3.30˜3.20 (m, 18H), 2.95˜2.90 (m, 1H), 2.05 (t, J=7.0 Hz, 2H). Td: 309° C.

The compound of Formula 1m: 1H NMR(CDCl₃, 400 MHz) 6.29 (s, 4H), 3.33(s, 6H), 2.10˜2.00 (m, 2H), 1.90˜1.80 (m, 4H), 1.70˜1.60 (m, 4H), 1.55˜1.35 (m, 4H).

The compound of Formula 1n: 1H NMR(CDCl₃, 400 MHz) 6.32 (s, 4H), 3.42 (s, 4H), 2.15˜2.05 (m, 2H), 1.90˜1.80 (m, 4H), 1.75˜1.65 (m, 4H), 1.40˜1.25 (m, 4H), 1.15 (bs, 6H).

The compound of Formula 1o: 1H NMR(CDCl₃, 400 MHz) 6.23 (s, 4H), 3.55˜3.40 (m, 8H), 3.31 (bs, 6H), 2.10˜2.00 (m, 2H), 1.90˜1.80 (m, 4H), 1.65˜1.55 (m, 4H), 1.35˜1.25 (m, 4H).

The compound of Formula 1p: 1H NMR(CDCl₃, 400 MHz) 6.21 (s, 2H), 5.45 (bs, 6H), 3.30˜3.20 (m, 12H), 2.80˜2.70 (m, 3H), 2.35˜2.25 (m, 2H), 1.80˜1.75 (m, 1H), 1.70˜1.60 (m, 4H), 1.50˜1.20 (bm, 24H), 1.01 (s, 3H).

The compound of Formula 1q: 1H NMR(CDCl₃, 400 MHz) 6.27 (s, 2H), 5.45 (bs, 6H), 3.50˜3.40 (m, 8H), 2.85˜2.75 (m, 3H), 2.35˜2.25 (m, 2H), 1.80˜1.70 (m, 1H), 1.55˜1.15 (bm, 37H).

The compound of Formula 1r: 1H NMR(CDCl₃, 400 MHz) 6.23 (s, 2H), 5.47 (bs, 6H), 3.50˜3.40 (m, 16H), 3.24 (bs, 12H), 2.35˜2.25 (m, 2H), 1.85˜1.75 (m, 1H), 1.75˜1.35 (bm, 28H), 1.05 (bs, 3H).

The compound of Formula 1s: 1H NMR(CDCl₃, 400 MHz) 5.69 (s, 24H), 3.55˜3.45 (m, 24H), 3.25 (t, J=4.9 Hz, 8H), 3.20˜3.10 (m, 8H), 2.85˜2.75 (m, 8H), 2.20˜2.15 (m, 8H), 1.65˜1.55 (m, 16H), 1.40˜1.30 (m, 20H), 1.11 (t, J=5.0 Hz, 12H).

### TEST EXAMPLE

### Activity Test of Photoresist Including Dissolution Promoter

1. NMR Comparison Test

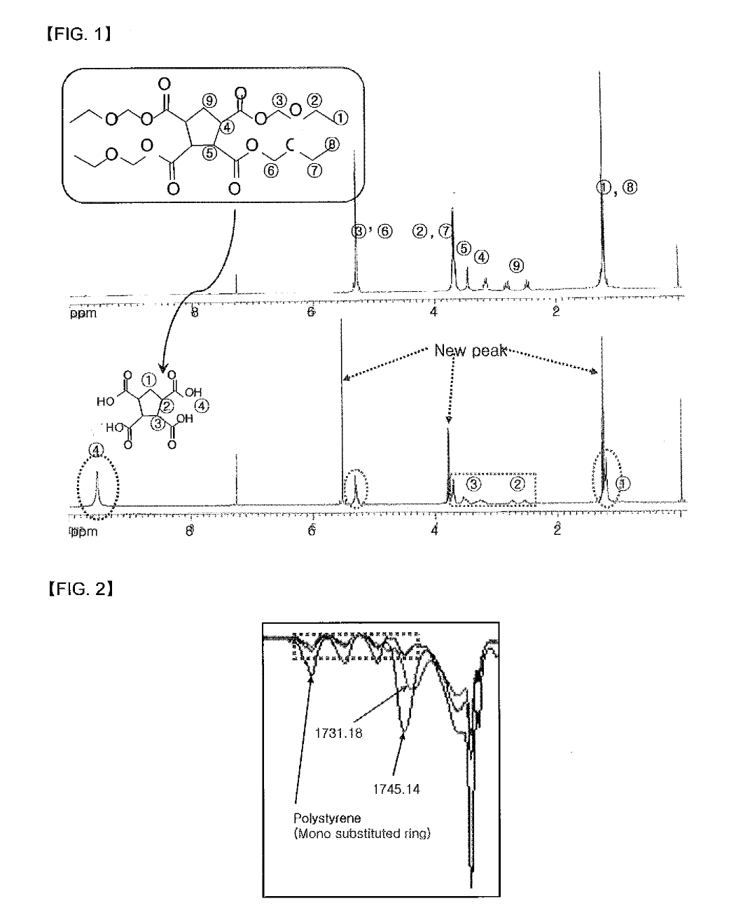

The following test was for verifying the deacetalization of the dissolution promoter in weak acid condition. First, the NMR data for the dissolution promoter represented by Formula 1h was obtained, and then 0.1 mg of triphenylsulfonium nonaflate as PAG diluted by 100 times with chloroform solvent substituted by deuterium was added in the dissolution promoter, and another NMR data was obtained and shown in the FIG. 1. As shown in the FIG. 1, if an acid is added, the peak from alcohol generated by the acid is the largest and clear, on the other hand, the peak from reactants are reduced. Moreover, 8 peaks from alcohol generated after the reaction are shown at 10 ppm. This explains that the dissolution promoter of the present invention is easily deprotected even in weak acid condition.

2. IR Comparison Test

IR intrinsic wavelength of carboxylic ester and carboxylic acid is 1750 and 1730 cm⁻¹. Intrinsic wavelength of carboxylic ester of the dissolution promoter and intrinsic wavelength of carboxylic acid of the sample deacetalized by light exposing process after adding acid in the dissolution promoter, are detected by IR, and shown in the FIG. 2. Concretely, 10 g of polystyrene (molecular weight: 5840, PDI(polydispersity index): 1.40) is dissolved in 10 g of propyleneglycol monomethyletheracetate (PGMEA), and evenly coated on a silicone wafer, and then intrinsic wavelength of polystyrene is obtained by IR test. Next, after coating the solution made by 1 g of the dissolution promoter (Formula 1h), 0.5 g of triphenylsulfonium nonaflate and the polystyrene solution on a silicone wafer, intrinsic wavelength of carboxylic ester is obtained by IR test, and then another IR test is proceeded after light exposing process of the coated silicone wafer. As the result, the peak from carboxylic ester is removed and the peak from carboxylic acid is generated, which indicates that acetal linked to carboxylic ester of the dissolution promoter during light exposing process is deprotected and detached as carboxylic acid. Therefore, as shown in the FIG. 2, in the case of the polymer without the dissolution promoter (shown as black line), there is no peak from carboxylic ester, but in the case of the polymer with the dissolution promoter (shown as blue line), a peak is shown at 1750 cm⁻¹ of intrinsic wavelength of carboxylic ester, and in the case of the deacetalized sample (shown as red line), there is no peak from carboxylic ester, and a peak is shown at 1730 cm⁻¹ of intrinsic wavelength of carboxylic acid. This explains that the dissolution promoter of the present invention is easily deprotected even in weak acid condition.

3. Difference of Solubility Rate After Light Exposure

As shown in the Table 2, a photoresist layer is formed on a silicone wafer using polymers for conventional KrF and ArF. The structure of the polymer for KrF is

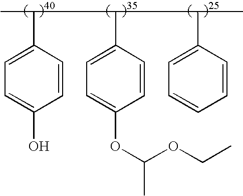

and the structure of the polymer for ArF is

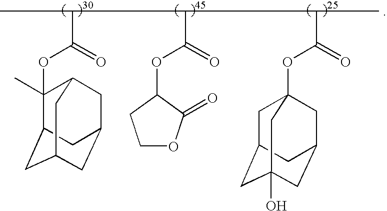

Moreover, while the photoresist layer is formed at the same thickness and conditions, the dissolution promoter as the component and content listed in the following Table 2, or the deacetalized dissolution promoter reactant was added in the polymer. After developing each photoresist layer, the thickness of the remaining photoresist layer is measured and shown in the Table 2.

TABLE 2

| | | Thickness | Thickness | |
|---|---|---|---|---|
| | Type of dissolution promoter and content | before | after | Dissolution |
| | with respect to | development | development | rate |
| Type of polymer | 100 weights parts of polymer | (Å) | (Å) | (Å/min) |
| Polymer for KrF | — | 8230 | 7610 | 620 |
| Polymer for KrF | Formula 2c, 5 weights part | 8022 | 0 | 8022 |
| Polymer for KrF | Formula 1b, 5 weights part | 8074 | 7427 | 647 |
| Polymer for KrF | Formula 3a, 5 weights part | 7910 | 0 | 7910 |
| Polymer for KrF | Formula 1h, 5 weights part | 7999 | 7361 | 638 |
| Polymer for KrF | Formula 1h, 10 weights part | 8820 | 8197 | 623 |
| Polymer for KrF | Formula 1i, 5 weights part | 8245 | 7589 | 656 |
| Polymer for KrF | Formula 5a, 5 weights part | 8730 | 7040 | 1690 |
| Polymer for KrF | Formula 5a, 10 weights part | 9377 | 4050 | 5327 |
| Polymer for KrF | Formula 1p, 5 weights part | 8820 | 8189 | 631 |
| Polymer for KrF | Formula 1p, 10 weights part | 9220 | 8440 | 780 |
| Polymer for ArF | — | 2969 | 2965 | 4 |
| Polymer for ArF | Formula 3a, 5 weights part | 3783 | 3581 | 202 |
| Polymer for ArF | Formula 1h, 5 weights part | 3781 | 3776 | 5 |

As shown in Table 2, after development, the thickness of photoresist layer is reduced as the amount of the deacetalized dissolution promoter reactant increases, on the other hand, in the case that the undecomposed dissolution promoter is added, after development, the difference in thickness of photoresist layers is not large. Therefore, the solubility of the photoresist layer is improved as the dissolution promoter reacts and is decomposed in the photoresist layer.

### EXAMPLES 20 TO 21, COMPARATIVE EXAMPLE

### Formation and Evaluation of Photoresist Pattern

A photoresist composition was produced by mixing 1 g of a photoresist polymer for conventional ArF represented by the following Formula 6 (wherein, R1, R2 and R3 are a hydrogen atom or a methyl group independently, R′ is a methyl group, and a:b:c=30 mol %:45 mol %:25 mol %), 0.05 g of triphenylsulfonium nonaflate as PAG, 0.02 g of triethylamine as base, 14 g of PGMEA as organic solvent, and the dissolution promoter of the component and amount listed in the following Table 3. After forming a photoresist pattern using the photoresist composition, the sensitivity, FCCD (First collapse critical dimension), and LER (line edge roughness) of the photoresist pattern were measured and are shown in the following Table 3, and the electron microphotograph images of the photoresist patterns are shown in FIG. 3 to 6.

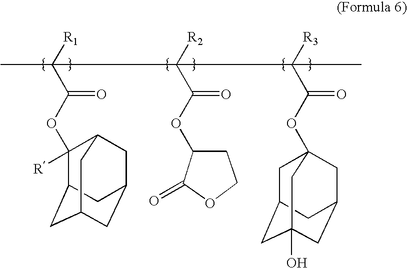

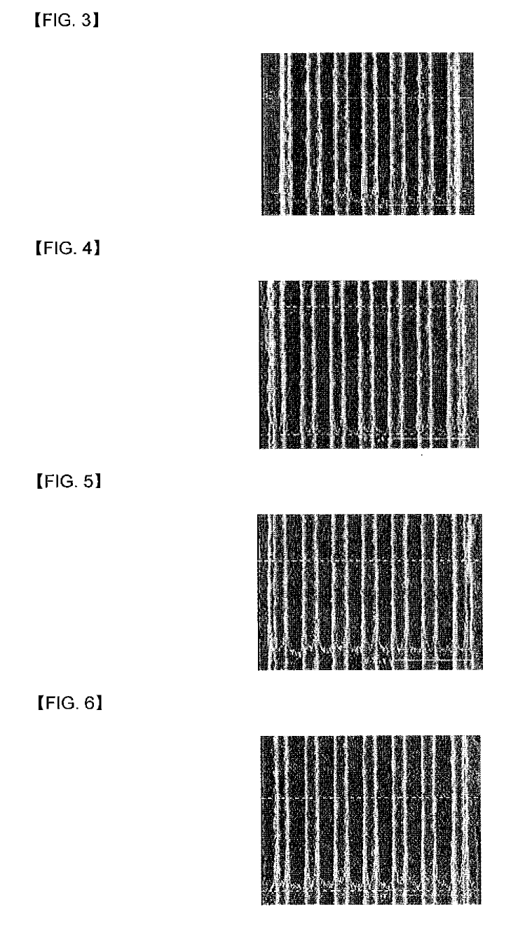

TABLE 3

| | Dissolution | Sensitivity | | LER (nm, | |
|---|---|---|---|---|---|
| | promoter | (mJ/cm²) | FCCD (nm) | 3sigma) | Drawings |
| Comparative Example | 0 g | 26 | 69.1 | 7.13 | FIG. 3 |
| Example 20 | Formula 1a, 0.02 g | 28 | 68.1 | 4.96 | FIG. 4 |
| Example 21 | Formula 1h, 0.02 g | 26 | 67.8 | 5.16 | FIG. 5 |
| Example 22 | Formula 1p, 0.02 g | 34 | 68.6 | 6.18 | FIG. 6 |

As shown in the Table 3, in the case of using the dissolution promoter of the present invention, the LER of the photoresist pattern is improved, and other properties of the photoresist pattern are same as or better than conventional photoresist composition.

As stated above, the dissolution promoter according to the present invention can increase the solubility of polymer to developer, and can be deprotected with a little amount of acid. Thus, the difference of solubility between exposed region and unexposed region can be increased effectively. Moreover, the photoresist composition according to the present invention can increase the contrast of photoresist pattern, and can prevent the formation of various defects and bridges during development process, and improve the line edge roughness.

1. A dissolution promoter having a structure of following Formula 1, 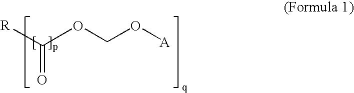
wherein, R is a hydrocarbon group of 1 to 40 carbon atoms, A is an alkyl group of 1 to 10 carbon atoms, p is 0 or 1, and q is an integer of I to 20.

2. The dissolution promoter of claim 1, wherein, R is a hydrocarbon group of 4 to 30 carbon atoms comprising cycloalkyl group or multi cycloalkyl group.

3. The dissolution promoter of claim 2, wherein, R is a ring structure comprising a hetero atom, and A is selected from a group consisting of —CH₃, —CH₂CH₃, —CH₂CH₂OCH₃, and —CH₂CH₂OCH₂CH₃.

4. The dissolution promoter of claim 1, wherein, the dissolution promoter is selected from a group consisting of include

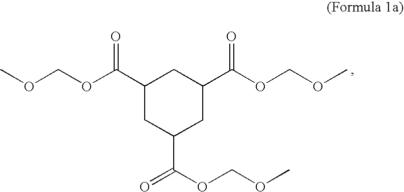

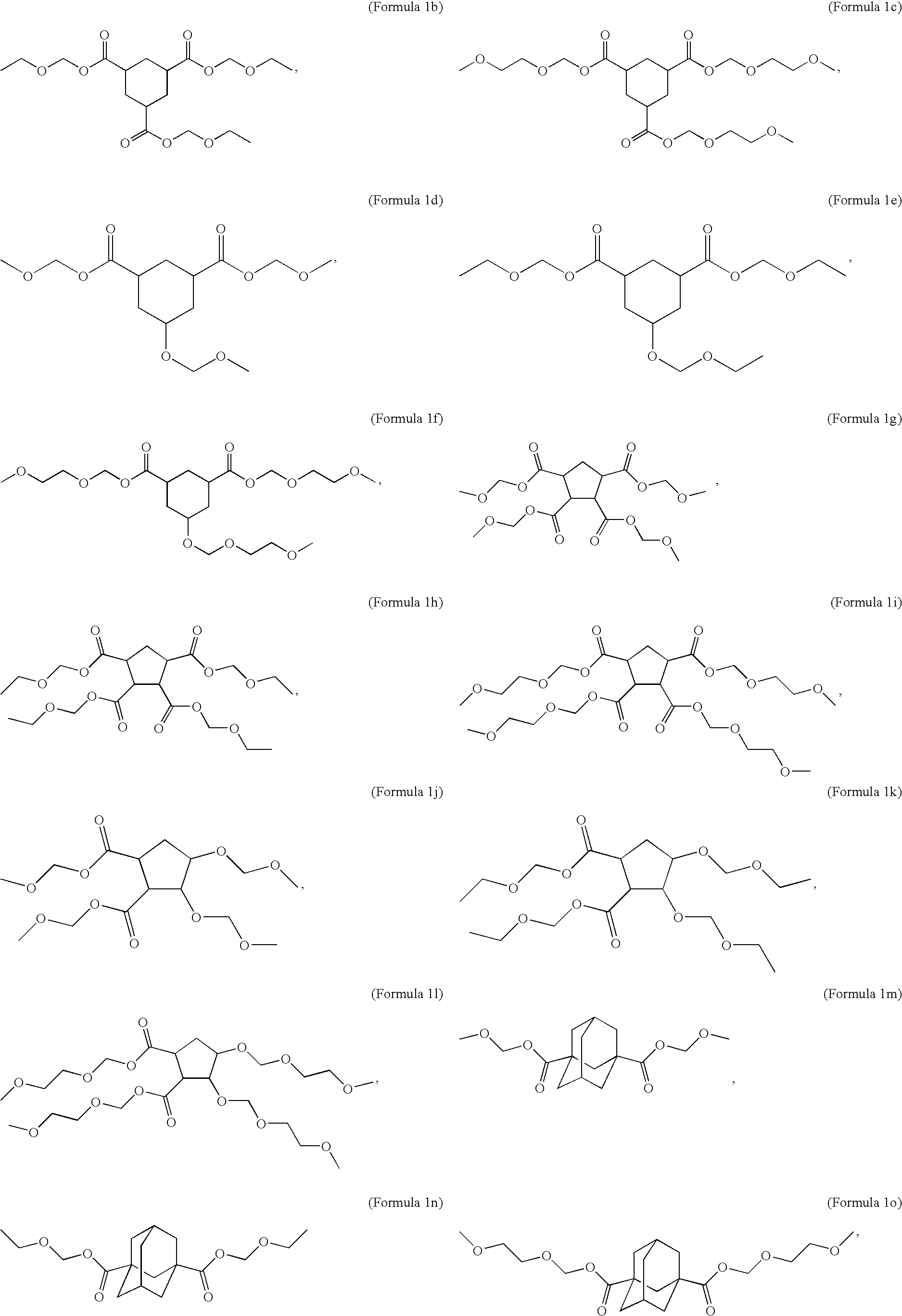

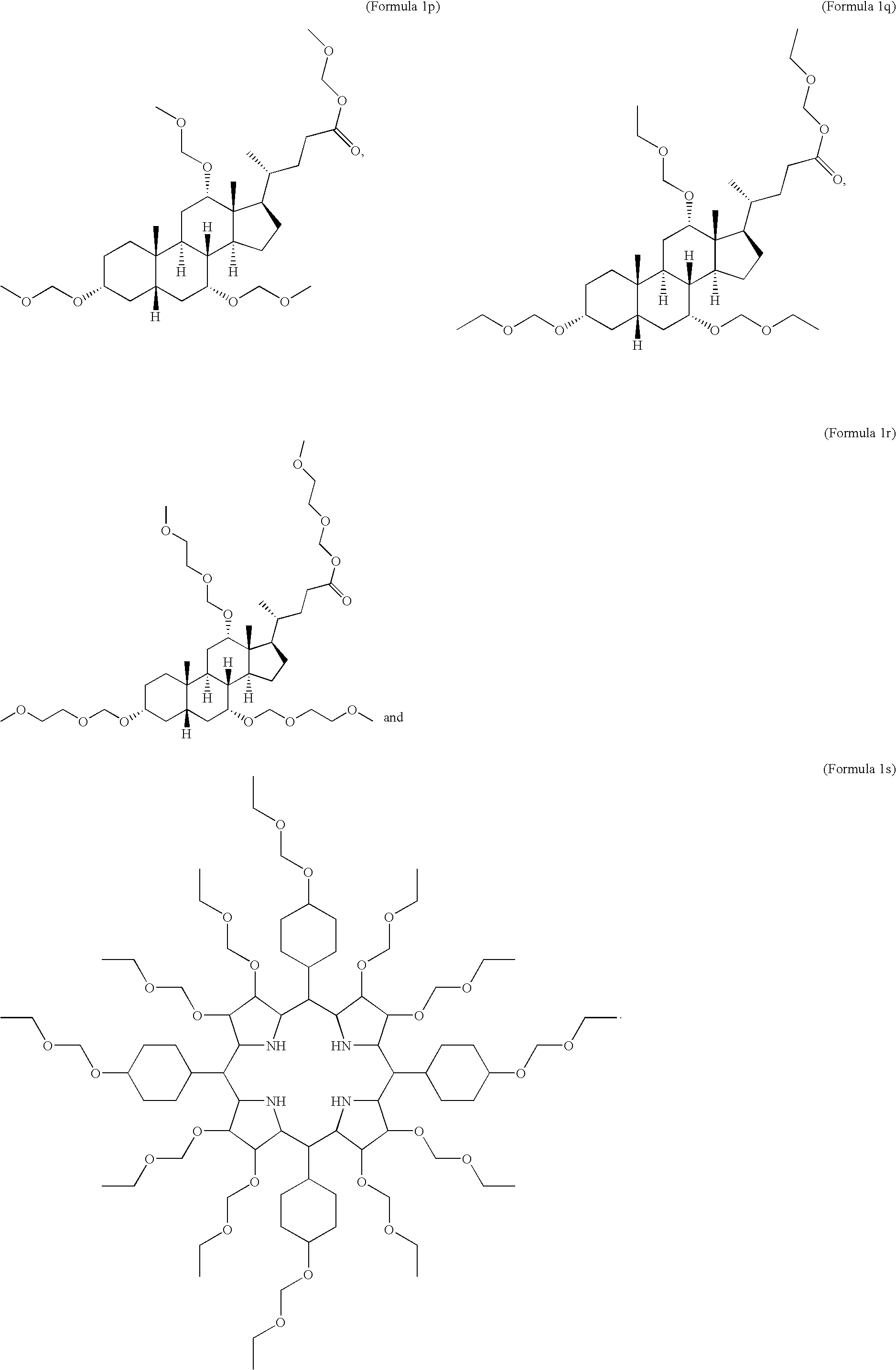

5. The dissolution promoter of claim 1, wherein, the dissolution promoter is obtained by reacting alcohol or carboxylic acid and alkyl halide, as shown in following Reaction 1, 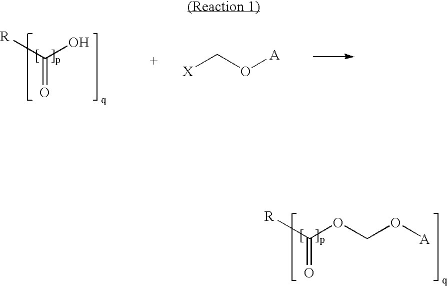
wherein, R, A, p and q are the same as defined in the Formula 1, and X is a halogen atom.

6. A photoresist composition comprising:
3 to 30 wt % (weight %) of photosensitive polymer;
1 to 30 weight parts of a dissolution promoter represented by following Formula 1, with respect to 100 weight parts of the photosensitive polymer, 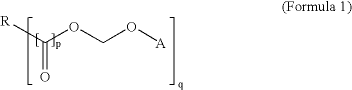
wherein, R is a hydrocarbon group of 1 to 40 carbon atoms, A is an alkyl group of 1 to 10 carbon atoms, p is 0 or 1, q is an integer of 1 to 20;
0.05 to 10 weight parts of a photo-acid generator with respect to 100 weight parts of the photosensitive polymer; and
a remaining organic solvent.

7. The photoresist composition of claim 6, wherein, the photosensitive polymer has a protecting group which is sensitive to an acid and then is separated by the acid.

8. The photoresist composition of claim 6, wherein, the photoresist composition further comprises 0.01 to 10 weight parts of a base compound with respect to the total photoresist composition, and 0.001 to 2 weight parts of a surfactant with respect to 100 weight parts of the solid content of the photoresist composition.

9. A method for forming a photoresist pattern, comprising the step of:
(a) coating a photoresist composition on a substrate to form a photoresist layer;
(b) exposing the photoresist layer to a light;
(c) heating the exposed photoresist layer; and
(d) developing the heated photoresist layer to form the desired photoresist pattern,
wherein, the photoresist composition comprises (i) 3 to 30 wt % of photosensitive polymer; (ii) 1 to 30 weight parts of a dissolution promoter represented by following Formula 1, with respect to 100 weight parts of the photosensitive polymer, 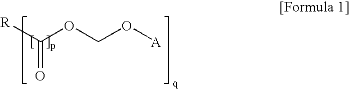
wherein, R is a hydrocarbon group of 1 to 40 carbon atoms, A is an alkyl group of 1 to 10 carbon atoms, p is 0 or 1, q is an integer of 1 to 20;
(iii) 0.05 to 10 weight parts of a photo-acid generator with respect to 100 weight parts of the photosensitive polymer; and (iv) a remaining organic solvent.
